(19) United States
(12) Patent Application Publication
(10) Pub. No.: US 2007/0135723 A1
(43) Pub. Date: Jun. 14, 2007

(54) Method for measuring a user's cardiorespiratory endurance by a fitness equipment

(76) Inventor: Leao Wang, Taiping (TW)

Correspondence Address: KUO-HSIUNG CHIU, 13F., NO.23, JIUN-HO STREET, PEITUN DISTRICT, TAICHUNG, 406, TW

(21) Appl. No.: 11/296,622

(22) Filed: Dec. 8, 2005

(51) Int. Cl. A61B 5/04

(52) U.S. Cl. 600/520

(57) ABSTRACT

The present invention relates to a method for measuring a user's cardiorespiratory endurance by a fitness equipment, which uses a fitness equipment such as an electric treadmill having a speed control meter panel and a heartbeat monitor to operate with an optimal human heartbeat rate (which is 60%˜90% of our maximum heartbeat rate), a basic exercise safe heartbeat rate (which is 40%˜60% of our maximum heartbeat rate) and a predetermined mid-strength testing exercise procedure for easily measuring the reference value of the cardiorespiratory endurance (also known as cardiorespiratory function) of a user at the time of the measurement, so as to provide the user a reference of physical conditions and a basis for future physical trainings and exercises.

### BACKGROUND OF THE INVENTION

1. Fields of the Invention

The invention relates to a method for measuring a user's cardiorespiratory endurance by a fitness equipment, and more particularly to a method for obtaining a reference value of the user's cardiorespiratory endurance by a general fitness equipment having a specific function, such that users can know about their physical conditions or use the reference value as a basis for further physical training.

2. Description of the Related Art

As we all know, the cardiorespiratory endurance is a major factor for determining our physical conditions, and thus knowing about one's cardiorespiratory functions and conditions by a popular fitness equipment and a simple procedure definitely can alert us about our health conditions and remind us to improve our physical conditions and further maintain our health. Similarly, the physical conditions absolutely affect the overall performance of a professional athletic, and thus professional athletics are trained to improve their cardiorespiratory endurance, which such training is an important part for maintaining their professional performance.

However, up to now, there is no fitness equipment having the foregoing function of measuring our cardiorespiratory endurance in a simple manner. Therefore, people regardless of the general public or professional athletics have to go to hospitals, related research institutes, or professional sport physiological laboratories for the physical examinations using expensive cardiorespiratory testing devices or related equipments. In addition to the costs, such arrangement also causes inconvenience to users.

As to most users, they maintain their physical strength and functions by a so-called “progressive self training” and determine whether or not their physical strength and functions make improvements or breakthroughs by their subjective recognition and self-conscience. However, such subjective inspection is simply a conscious evaluation without strong supports by scientific data, and thus easily causing distortion or discrepancy. Since our cardiorespiratory endurance varies with many subjective and objective factors (such as the level of the examinee's physiological tiredness, psychological conditions, external environment, and climatic factors, etc). If there is no definite support of scientific data, the testing results will be questionable, and people will lose their benefits of having positive self-training.

### SUMMARY OF THE INVENTION

Therefore, it is a primary object of the invention to provide an existing fitness equipment having a specific operating functions (such as the model that comes with a speed control meter and a built-in program for entering the user's age, and automatically calculating the optimal heartbeat rate, basic exercise safe heartbeat rate, and a heartbeat monitor) to operate with a simple mid-strength testing exercise procedure for effectively obtaining the best reliable reference value of the cardiorespiratory endurance, and further effectively maximizing the utility, value, and significance of the fitness equipment. To each user, regardless of professional athletics or general public, such fitness equipment can be used to provide the information about the user's physical function and conditions and assure the user's health conditions, so as to maintain and improve our cardiorespiratory endurance.

Therefore, the present invention mainly uses a fitness equipment such as an electric treadmill having a speed control meter panel and a heartbeat monitor to operate with the optimal human heartbeat rate (which is 60%˜90% of our maximum heartbeat rate), the basic exercise safe heartbeat rate (w hich is 40%˜60% of our maximum heartbeat rate) and a predetermined mid-strength testing exercise procedure for easily measuring the reference value of the cardiorespiratory endurance (also known as cardiorespiratory function) of a user at the time of the measurement, so as to provide the user a reference of physical conditions and a basis for future physical training and exercise.

### BRIEF DESCRIPTION OF THE DRAWINGS

The accomplishment of this and other objects of the invention will become apparent from the following description and its accompanying drawings of which:

FIG. 1 is a flow chart of a testing exercise procedure according to a preferred embodiment of the present invention;

FIG. 2 is a list of measuring results of a first examinee according to a preferred embodiment of the present invention; and

FIG. 3 is a list of measuring results of a second examinee according to a preferred embodiment of the present invention.

### DETAILED DESCRIPTION OF THE PREFERRED EMBODIMENT

Before the testing method in accordance with the present invention is described, some of the terminologies used in this specification are explained first.

Maximum Heartbeat Rate: It refers to the maximum heartbeat that a human body can “possibly” bear. At present, both medical and academic areas accept 220 times per minute as the maximum heartbeat rate.

Optimal Heartbeat Rate: It is equal to the maximum heartbeat rate subtracted by the examinee's age and then multiplied by 60%˜90%. At present, both medical and academic areas accept such reference value as the optimal heartbeat rate.

Basic Exercise Safe Heartbeat Rate: It is equal to the maximum heartbeat rate subtracted by the examinee's age and then multiplied by 40%˜60%. At present, both medical and academic areas accept such reference value as the optimal heartbeat rate.

Monitoring Meter Panel: It is an electronic meter panel for monitoring the exercise speed, distance, and related parameters of a fitness equipment, and capable of displaying the parameters on a specific window of the panel. Most of the present fitness equipments come with this device.

Heartbeat Monitor: It is a device being in contact with an examinee for detecting the heartbeats of the examinee anytime, and capable of display the heartbeats on a specific window of the speed control meter panel. Most of the present fitness equipments come with this device.

Cardiorespiratory Endurance Value (abbreviated as RSV HRC): The RSV HRC value finally measured by the method of the present invention is based on 60%˜90% of the optimal heartbeat rate (and the optimal heartbeat rate is used as the continuous load limit).

Testing Exercise: It could be the jogging exercise on a treadmill, pedaling exercise on a fitness bike, or simulated running exercise of an elliptical trainer, depending on the type of the fitness equipment.

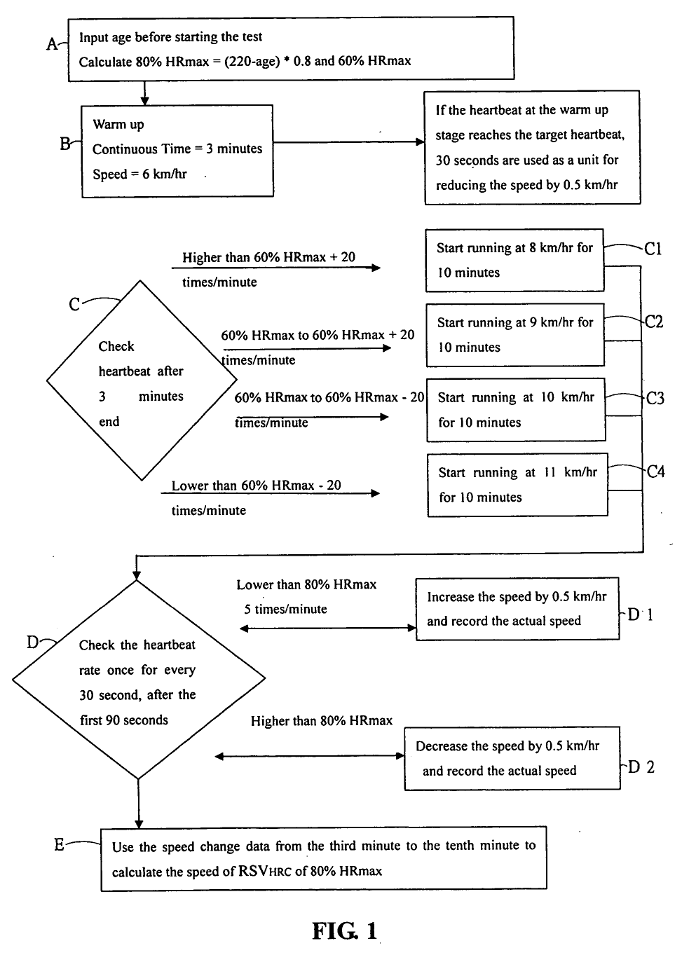

Now, referring to FIG. 1 for the flow chart of the testing exercise procedure according to a preferred embodiment of the present invention, this preferred embodiment adopts 80% of the optimal heartbeat rate, and 60% of the basic exercise safe heartbeat, and 10 minutes of the exercise time as the basis of the testing conditions, and the procedure comprises the following steps:

Step A: Enter the user's age into the monitoring meter panel of a fitness equipment, and a built-in program of the monitoring meter panel calculates the user's 80% of the optimal heartbeat rate value and 60% of the basic exercise safe heartbeat rate value;

Step B: Allow the user to warm up continuously for three minutes, and set the exercise speed to 6 Km/hr, and progressively lower the exercise speed if the user's heartbeat value is too large;

Step C: Check the heartbeat value;

Step C1: Start a mid-strength testing exercise at the speed of 8 Km/hr for 10 minutes, if the measured heartbeat value is higher than the sum of the basic exercise safe heartbeat rate value and 20 times/minute;

Step C2: Start a mid-strength testing exercise at the speed of 9 Km/hr for 10 minutes, if the measured heartbeat value is higher than the sum of the basic exercise safe heartbeat rate value and 20 times/minute;

Step C3: Start a mid-strength testing exercise at the speed of 10 Km/hr for 10 minutes, if the measured heartbeat value is higher than the sum of the basic exercise safe heartbeat rate value and 20 times/minute;

Step C4: Start a mid-strength testing exercise at the speed of 11 Km/hr for 10 minutes, if the measured heartbeat value is higher than the sum of the basic exercise safe heartbeat rate value and 20 times/minute;

Step D: From the first 90 seconds on, check the heartbeat value once for every 30 seconds and record the heartbeat value;

Step D1: Increase the speed by 0.5Km/hr if the measured heartbeat value is 5 times/minute lower than the optimal heartbeat value, and record the speed;

Step D2: Decrease the speed by 0.5Km/hr if the measured heartbeat value is higher than the optimal heartbeat value, and record the speed;

Step E: Complete the 16-minute testing exercise and substitute the actual speed reading from the third minute to the tenth minute into the following formula to obtain the RSV HRC value.

The formula for calculating RSV HRC value is given below:

$$RSV HRC = \frac{Z ( d - Md ) \times ( T - MT )}{{Z ( T - MT )}^{2}}$$

Where, d-Md is an average difference between the exercise distances;
T-MT is the average difference between the exercise time; and
z is the sum.

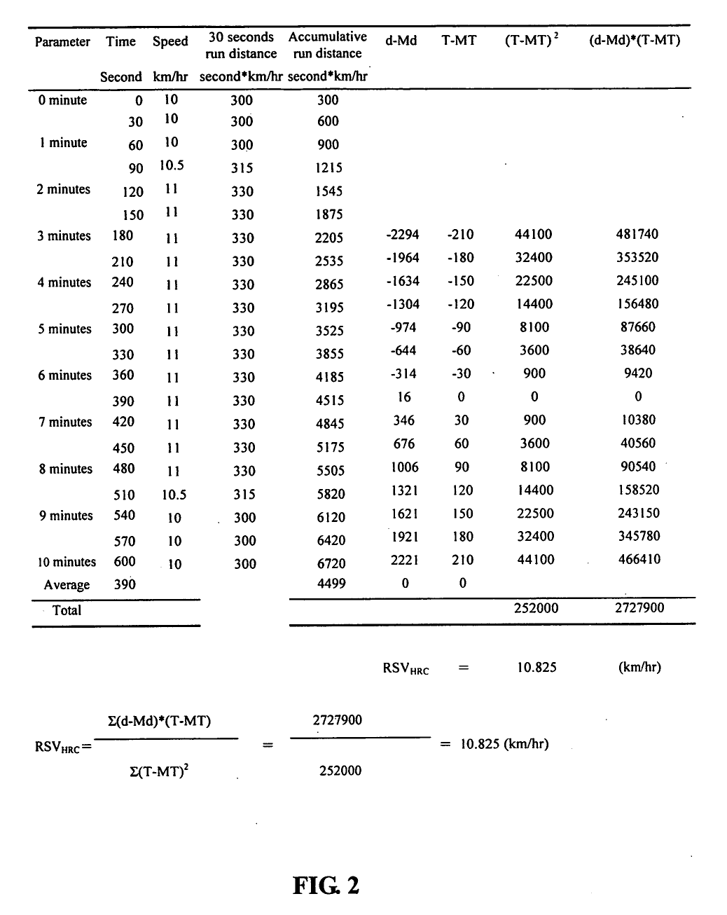

Referring to FIG. 2 for the list of measuring results of a first examinee (who uses an electric treadmill as the testing model), the examiner can have a better understanding about the operation of the formula. The average time used by the first examinee is 390 (which is a constant), and the average exercise distance is 4499 (which is usually a variable). Therefore, the T-MT value at the third minute is equal to 80−390=−210; the d-Md value is equal to 2205−4499=−2294; and so no, and (T-MT)² is equal to −210×−210=44100, and (d-Md)×(T-MT) is equal to −2294×−210=481740.

The RSV HRC value equals to the distance difference multiplied by the sum of time differences and divided by the sum of squares of the time differences, as follows:
2727900÷252000=10.825(km/hr)

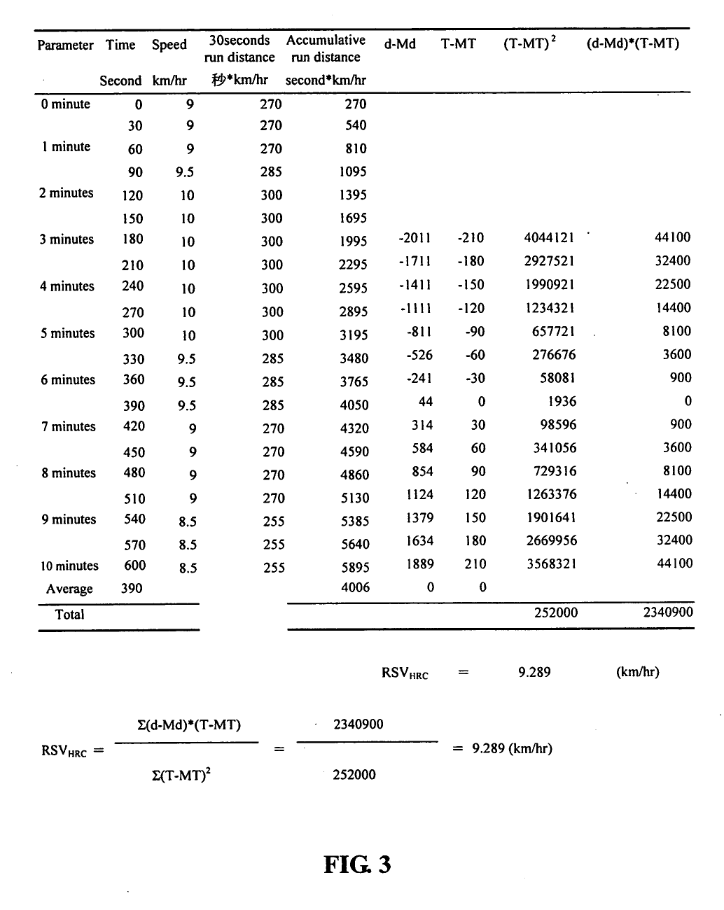

Referring to FIG. 3 for the list of measuring results of a second examinee compared with the list of measuring results of the first examinee shown in FIG. 2, the higher the examinee's RSV HRC value, the better is the examinee's cardiorespiratory endurance. In other words, the exercise distance becomes larger and the exercise speed becomes faster per same testing unit time. The lower the heartbeat value, the better is the cardiorespiratory function. This testing method has been used for testing several tens of examinees to obtain the actual test results, and the test results showed that this method has high reliability and it is definitely a practical and useful testing method.

The inventor of the present invention adopted the so-called “Basic Exercise Safe Heartbeat Rate” as the standard for measuring the heartbeat and constant exercise speed after the warm up, because the heartbeat value will increase and accelerate according to the level of exercise when a person is doing an aerobic exercise. Based on the safety factor, the present invention designs a “Safety Heartbeat Standard” to prevent an overload of the examinee's heartbeat. More particularly, such arrangement provides a protection for people whose cardiorespiratory function have been damaged seriously, elderly people, or people having a poor cardiorespiratory function. Let us take an examinee of age 20 and an examinee of age 60 for examples as follows:
(220−20)×60%=120 times/minute
(220−60)×60%=96 times/minute

From the above expressions, examinees of different ages have a small difference between their basic exercise safe heartbeat rate values, and thus the present invention uses such difference as the standard for starting the measurements of heartbeats and constant exercise speeds. Further, the calculation of using positive and negative 20 times/minute is adopted to accurately distinguish the examinees with a special physical capability (either having a very good physical capability or having a very poor physical capability) to obtain a safer and gentler testing procedure.

Further, the so-called “Mid-Strength Exercise” refers to a “Non-Exhausted Exercise” used for measuring the cardiorespiratory endurance reference value, primarily emphasizing on making this method applicable for each people who uses the fitness equipment, regardless of male, female, elderly, or youth. As described above, a more precise professional equipment or device is needed for measuring the actual cardiorespiratory endurance value of each examinee, and the examinee has to try very hard to exercise until his/her strength is exhausted before an accurate cardiorespiratory value can be obtained. Further, the same testing procedure must be repeated several times to obtain a more definite and truer testing data. However, most of the examinees do not have to go through such a violent and potentially dangerous testing procedure, but just want to have a general idea about their current physical conditions by using the fitness equipment. Furthermore, examinees should not do excessively violent exercises without being accompanied by professional trainers or medical personnel. Therefore, the testing procedure and a gentler mid-strength exercise in accordance with the present invention can assure the safety of the examinees.

With the same testing procedure and RSV HRC formula, the present invention operates with different optimal heartbeat rate values, basic exercise safe heartbeat rate values, and exercise time (such as the standard of using 90% for the optimal heartbeat rate, 50% for the basic exercise safe heartbeat rate, and 15 minutes for the exercise time) to measure the RSV HRC reference value. However, the data or parameters used as the measuring standard for obtaining the best testing reference value relies on our actual practices, experiments, and verifications. Obviously, these standards will not affect the validity, usefulness, and convenience of the present invention.

In summation of the above description, the present invention herein enhances the performance than the conventional structure and further complies with the patent application requirements and is submitted to the Patent and Trademark Office for review and granting of the commensurate patent rights.

What is claimed is:

1. A method for measuring a user's cardiorespiratory endurance by a fitness equipment, comprising the steps of using a fitness equipment having a speed control meter panel and a heartbeat monitor to operate with an optimal heartbeat rate, a basic exercise safe heartbeat rate, and a predetermined mid-strength testing exercise procedure for easily measuring said user's cardiorespiratory reference value (RSV HRC) at the time of measurement.

2. The method of measuring a user's cardiorespiratory endurance by a fitness equipment of claim 1, wherein said optimal heartbeat rate is defined as the heartbeat rate falling in the range of 60%˜90% of said optimal heartbeat rate.

3. The method of measuring a user's cardiorespiratory endurance by a fitness equipment of claim 1, wherein said basic exercise safe heartbeat rate is defined as the heartbeat rate falling in the range of 40%˜60% of said optimal heartbeat rate.

4. The method of measuring a user's cardiorespiratory endurance by a fitness equipment of claim 1, wherein said RSV HRC value is calculated by the formula:
$$RSV HRC = \frac{Z ( d - Md ) \times ( T - MT )}{{Z ( T - MT )}^{2}}$$
where, said d-Md is equal to an average difference of exercise distances; T-MT is equal to an average difference of exercise time; and z is equal to the total.

5. A method for measuring a user's cardiorespiratory endurance by a fitness equipment, comprising the step of:
(A) entering the user's age into a monitor meter panel, and a built-in program of said monitor meter panel calculating a user's predetermined optimal heartbeat rate value and a predetermined basic exercise safe heartbeat rate value;
(B) allowing said user to warm up continuously for at least three minutes, and setting an exercise speed to 6 Km/hr, and progressively reducing said exercise speed if the heartbeat value of said user is too large;
(C) checking said heartbeat value;
(C1): starting a mid-strength testing exercise at the speed of at least 8 Km/hr for at least ten minutes, if the measured heart value is larger than the sum of said basic exercise safe heartbeat value and 20 times/minute;
(C2) starting a mid-strength testing exercise at the speed of at least 9 Km/hr for at least ten minutes, if the measured heart value is larger than the sum of said basic exercise safe heartbeat value and 20 times/minute;
(C3) starting a mid-strength testing exercise at the speed of at least 10 Km/hr for at least ten minutes, if the measured heart value is larger than the sum of said basic exercise safe heartbeat value and 20 times/minute;
(C4) starting a mid-strength testing exercise at the speed of at least 11 Km/hr for at least ten minutes, if the measured heart value is larger than the sum of said basic exercise safe heartbeat value and 20 times/minute;
(D) examining a heartbeat value once for every 30 seconds starting for the first 90 seconds, and recording said heartbeat value;
(D1) increasing said exercise speed for at least 0.5 Km/hr, if said measured heartbeat value is lower than said optimal heartbeat value by 5 times/minute, and recording said exercise speed;
(D2) decreasing said exercise speed for at least 0.5 Km/hr, if said measured heartbeat value is higher than said optimal heartbeat value, and recording said exercise speed;
(E) completing said testing exercise in a predetermined unit time, and inputting actual exercise speeds measured from the third minute to the end of said exercise into a formula to obtain said RSV HRC value.

6. The method of measuring a user's cardiorespiratory endurance by a fitness equipment of claim 5, wherein said optimal heartbeat rate is defined as the heartbeat rate falling in the range of 60%˜90% of said optimal heartbeat rate.

7. The method of measuring a user's cardiorespiratory endurance by a fitness equipment of claim 5, wherein said basic exercise safe heartbeat rate is defined as the heartbeat rate falling in the range of 40%˜60% of said optimal heartbeat rate.

8. The method of measuring a user's cardiorespiratory endurance by a fitness equipment of claim 5, wherein said RSV HRC value is calculated by the formula:
$$RSV HRC = \frac{Z ( d - Md ) \times ( T - MT )}{{Z ( T - MT )}^{2}}$$
where, said d-Md is equal to an average difference of exercise distances; T-MT is equal to an average difference of exercise time; and z is equal to the total..
